(19) United States
(12) Patent Application Publication
(10) Pub. No.: US 2024/0217998 A1
(43) Pub. Date: Jul. 4, 2024

(54) Diphosphites with a dicyclohexylphosphino radical

(71) Applicant: Evonik Oxeno GmbH & Co. KG, Marl (DE)

(72) Inventors: Anna Chiara Sale, Recklinghausen (DE); Robert Franke, Marl (DE); Dirk Fridag, Haltern am See (DE); Peter Kucmierczyk, Herne (DE); Ana Markovic, Haltern am See (DE); Armin Börner, Rostock (DE); Detlef Selent, Rostock (DE)

(73) Assignee: Evonik Oxeno GmbH & Co. KG, Marl (DE)

(21) Appl. No.: 18/541,611

(22) Filed: Dec. 15, 2023

(30) Foreign Application Priority Data
Dec. 27, 2022 (EP) 22216763.7

(51) Int. Cl. C07F 9/50; B01J 31/18; B01J 31/22; C07C 45/50

(52) U.S. Cl. CPC C07F 9/5018; B01J 31/1885; B01J 31/2234; B01J 31/2295; C07C 45/50; B01J 2231/321; B01J 2531/822

(57) ABSTRACT

Diphosphites with a dicyclohexylphosphino radical and use thereof in hydroformylation.

The invention relates to diphosphites with a dicyclohexylphosphino radical and use thereof in hydroformylation.

Phosphorus-containing compounds play a crucial role as ligands in a multitude of reactions, e.g. in hydrogenation, in hydrocyanation and also in hydroformylation.

The reactions between olefin compounds, carbon monoxide and hydrogen in the presence of a catalyst to afford the aldehydes comprising one more carbon atom are known as hydroformylation or the oxo process. In these reactions, compounds of the transition metals of group VIII of the Periodic Table of the Elements are frequently employed as catalysts. Known ligands are, for example, compounds from the phosphine, phosphite and phosphonite classes, each containing trivalent phosphorus P^{III}. A good overview of the status of hydroformylation of olefins can be found in R. Franke, D. Selent, A. Börner, “Applied Hydroformylation”, Chem. Rev., 2012 (112), 11, 5675-5732, DOI:10.1021/cr3001803.

The technical object of the invention is to provide a compound with which an increased yield of aldehyde can be achieved in the hydroformylation of olefins.

The object is achieved by a compound according to claim 1.

Compound of formula (I):

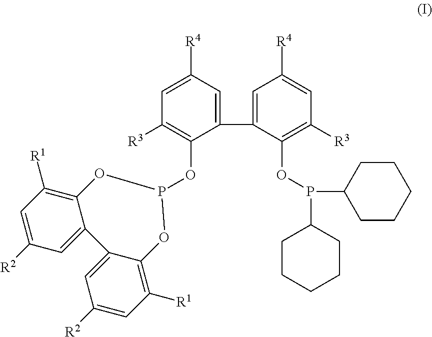

where R¹, R², R³, R⁴ are selected from: —(C₁-C₁₂)-alkyl, —O—(C₁-C₁₂)-alkyl.

In one embodiment, R¹ is selected from: —OCH₃, -^{tert}Bu.

In one embodiment, R¹ is -^{tert}Bu.

In one embodiment, R² is selected from: —OCH₃, -^{tert}Bu.

In one embodiment, R² is -^{tert}Bu.

In one embodiment, R² is —OCH₃.

In one embodiment, R³ is selected from: —OCH₃, -^{tert}Bu.

In one embodiment, R³ is -^{tert}Bu.

In one embodiment, R⁴ is selected from: —OCH₃, -^{tert}Bu.

In one embodiment, R⁴ is -^{tert}Bu.

In one embodiment, R⁴ is —OCH₃.

In one embodiment, the compound has the structure (1) or (2):

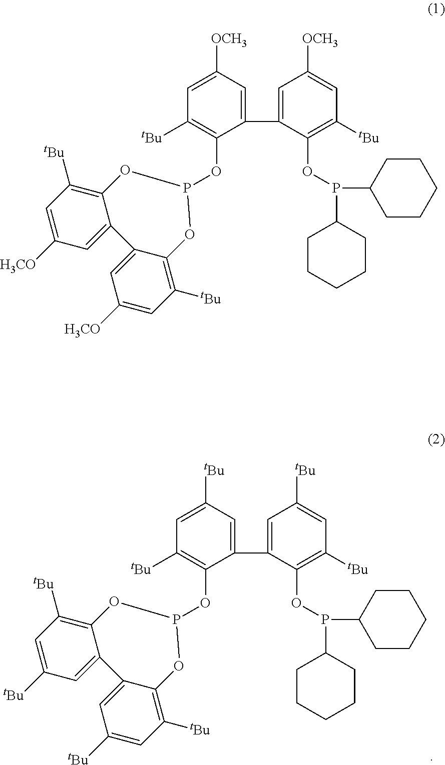 [SMILES: COc1cc(-c2cc(OC)cc(C(C)(C)C)c2Op2oc3c(C(C)(C)C)cc(OC)cc3c3cc(OC)cc(C(C)(C)C)c3o2)c(OP(C2CCCCC2)C2CCCCC2)c(C(C)(C)C)c1 ; CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP(C2CCCCC2)C2CCCCC2)c(C(C)(C)C)c1]

In one embodiment, the compound has the structure (1):

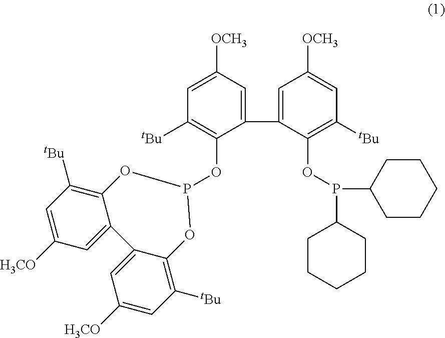 [SMILES: COc1cc(-c2cc(OC)cc(C(C)(C)C)c2Op2oc3c(C(C)(C)C)cc(OC)cc3c3cc(OC)cc(C(C)(C)C)c3o2)c(OP(C2CCCCC2)C2CCCCC2)c(C(C)(C)C)c1]

In one embodiment, the compound has the structure (2):

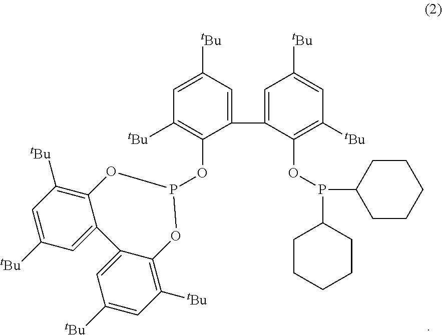 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP(C2CCCCC2)C2CCCCC2)c(C(C)(C)C)c1]

In addition to the compound itself, a process in which the compounds described above are used is also claimed.

Process comprising the process steps of:
a) initially charging an olefin;
b) adding a compound described above;
c) adding a Rh compound;
d) feeding in H₂ and CO;
e) heating the reaction mixture from a) to d), to convert the olefin to an aldehyde.

In one variant of the process, the Rh compound is selected from: Rh(acac)(CO)₂, [(acac)Rh(COD)] (Umicore, acac=acetylacetonate anion; COD=1,5-cyclooctadiene), Rh₄CO₁₂.

In one variant of the process, the Rh compound is Rh(acac)(COD).

In one variant of the process, the H₂ and CO in process step d) are fed in at a pressure in the range from 1 to 6 MPa (10 to 60 bar).

In one variant of the process, the H₂ and CO in process step d) are fed in at a pressure in the range from 1.5 to 4.5 MPa (15 to 45 bar).

In one variant of the process, the heating of the reaction mixture in process step e) is to a temperature within a range from 80° ° C. to 160° C.

In one variant of the process, the heating of the reaction mixture in process step e) is to a temperature within a range from 100° ° C. to 140° C.

The invention shall be elucidated in more detail hereinbelow with reference to working examples.

### Synthese (1): 4,8-Di-tert-butyl-6-((3,3′-di-tert-butyl-2′-((dicyclohexylphosphanyl)oxy)-5,5′-dimethoxy-[1,1′-biphenyl]-2-yl)oxy)-2,10-dimethoxydibenzo[d,f][1,3,2]dioxaphosphepine

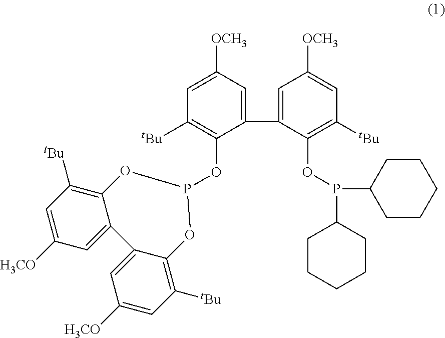 [SMILES: COc1cc(-c2cc(OC)cc(C(C)(C)C)c2Op2oc3c(C(C)(C)C)cc(OC)cc3c3cc(OC)cc(C(C)(C)C)c3o2)c(OP(C2CCCCC2)C2CCCCC2)c(C(C)(C)C)c1]

To a solution of 3,3′-di-tert-butyl-2′-((4,8-di-tert-butyl-2,10-dimethoxydibenzo[d,f][1,3,2]dioxaphosphepin-6-yl)oxy)-5,5′-dimethoxy-[1,1′-biphenyl]-2-ol (0.6749 g; 0.906 mmol) in THF (9 ml) is added dropwise at −20° C. a 0.533M solution of n-butyllithium (1.7 ml; 0.906 mmol) in hexane. The mixture is stirred for 20 min at this temperature, allowed to warm to 0° C. and a solution of chlorodicyclohexylphosphine (0.2214 g; 0.9513 mmol) in THF (4 ml) is added. The mixture is allowed to warm to room temperature, stirred overnight and the solvent removed in vacuo. The residue is taken up in toluene (9 ml), the resulting mixture is filtered, the filtrate concentrated in vacuo and dried at 50° C./0.1 mbar for 2 h. The residue obtained is crystallized from hot acetonitrile (8 ml). Filtration and vacuum drying gives 0.630 g (0.670 mmol, 74%).

Elemental analysis (calculated for C₅₆H₇₈O₈P₂=941.18 g/mol): C=71.35 (71.47); H=8.40 (8.35); P=6.42 (6.58)%.

ESI-TOF HRMS: m/z=941.5244; [M++H], calculated m/z=941.5250.

### Synthese (2): 2,4,8,10-Tetra-tert-butyl-6-((3,3′,5,5′-tetra-tert-butyl-2′-((dicyclohexylphosphanyl)oxy)-[1,1′-biphenyl]-2-yl)oxy)dibenzo[d,f][1,3,2]dioxaphosphepine

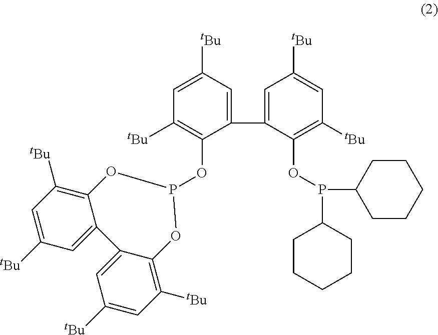 [SMILES: CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP(C2CCCCC2)C2CCCCC2)c(C(C)(C)C)c1]

To a solution of 3,3′,5,5′-tetra-tert-butyl-2′-((2,4,8,10-tetra-tert-butyldibenzo[d,f][1,3,2]dioxaphosphepin-6-yl)oxy)-[1,1′-biphenyl]-2-ol (0.4897 g; 0.5766 mmol) in THF (5 ml) is added dropwise at −20° C. a 0.533M solution of n-butyllithium (1.14 ml; 0.6054 mmol) in hexane. The mixture is stirred for 20 min at this temperature, allowed to warm to 0° C. and a solution of chlorodicyclohexylphosphine (0.1436 g; 0.6170 mmol) in THF (3 ml) is added. The mixture is allowed to warm to room temperature, stirred overnight and the solvent removed in vacuo. The residue is taken up in toluene (9 ml), the resulting mixture is filtered, the filtrate concentrated in vacuo and dried at 50° C./0.1 mbar for 2 h. The residue is dissolved in THF (3.5 ml). The solid precipitated after addition of acetonitrile (ca. 5 ml) is filtered, washed with a little acetonitrile and dried. Yield: 0.272 g (0.260 mmol, 45%).

Elemental analysis (calculated for C₆₈H₁₀₂O₄P₂=1045.50 g/mol): C=78.23 (78.12); H=9.74 (9.83); P=5.71 (5.93)%.

ESI-TOF HRMS: m/z=1045.7322; [M++H], calculated m/z=1045.7332.

### Synthese (3): ((3,3′,5,5′-Tetra-tert-butyl-[1,1′-biphenyl]-2,2′-diyl)bis(oxy)bis (dicyclohexylphosphane)

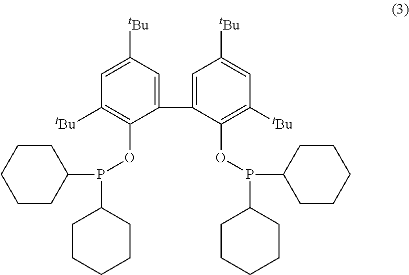 [SMILES: CC(C)(C)c1cc(-c2cc(C(C)(C)C)cc(C(C)(C)C)c2OP(C2CCCCC2)C2CCCCC2)c(OP(C2CCCCC2)C2CCCCC2)c(C(C)(C)C)c1]

To a solution of 3,3′,5,5′-tetra-tert-butyl-[1,1′-biphenyl]-2,2′-diol (0.6832 g; 1.6638 mmol) in THF (12 ml) is added dropwise at −20° ° C. a 0.533M solution of n-butyllithium (6.56 ml; 3.494 mmol) in hexane. The mixture is stirred for 20 min at this temperature, allowed to warm to 0° C. and a solution of chlorodicyclohexylphosphine (0.8287 g; 3.5606 mmol) in THF (6 ml) is added. The mixture is allowed to warm to room temperature, stirred overnight and the solvent removed in vacuo. The residue is taken up in toluene (20 ml), the resulting mixture is filtered, the filtrate concentrated in vacuo and dried at 50° C./0.1 mbar for 2 h. The residue is dissolved in THF (6 ml). The solid precipitated after addition of acetonitrile (4 ml) is filtered and dried. Yield: 0.469 g (0.582 mmol, 35%).

ESI-TOF HRMS: m/z=803.6021; [M++H]; calculated m/z=803.6025.

### Catalysis Experiments

The hydroformylation was conducted in a 200 ml autoclave from Premex Reactor AG, Lengau, Switzerland, equipped with pressure-retaining valve, gas flowmeter, sparging stirrer and pressure pipette. To minimize the influence of moisture and oxygen, the toluene used as solvent was purified in a Pure Solv. MD-7 system and stored under argon. The olefin cis/trans-2-pentene used as substrate (Aldrich) was heated at reflux over sodium and distilled under argon. Toluene solutions of the catalyst precursor and of the ligand were mixed in the autoclave under an argon atmosphere. [(acac)Rh(COD)] (Umicore, acac=acetylacetonate anion; COD=1,5-cyclooctadiene) was used as catalyst precursor. The autoclave was heated with stirring (1500 rpm) at 12 bar for a final pressure of 20 bar. After reaching the reaction temperature, the olefin was injected into the autoclave by way of a positive pressure established in the pressure pipette. The reaction was carried out at a constant pressure of 20 bar (closed-loop pressure controller from Bronkhorst, the Netherlands) over 4 h. At the end of the reaction time, the autoclave was cooled to room temperature, depressurized while stirring and purged with argon. 1 ml of reaction mixture was removed immediately after the stirrer had been switched off, diluted with 10 ml of pentane and analysed by gas chromatography: HP 5890 Series II plus, PONA, 50 m×0.2 mm×0.5 μm.

The experiment was carried out with compounds (1), (2) and (3).

The compound (3) serves here as reference ligand.

### Results of the Catalysis Experiments

[Rh]: 100 ppm, p: 20 bar, T: 120° C.; t: 4 h; Rh:L=1:2

TABLE
Hydroformylation of cis/trans-2-pentene

| Ligand | Aldehyde yield [%] |
|---|---|
| (1)* | 94 |
| (2)* | 99 |
| (3) | <1 |

*inventive compound

The experiments carried out demonstrate that the stated object is achieved by a compound according to the invention.

1. Compound of formula (I): 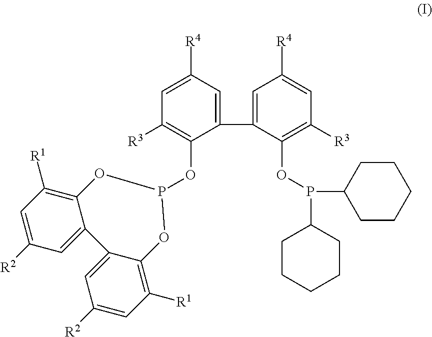
where R¹, R², R³, R⁴ are selected from: —(C₁-C₁₂)-alkyl, —O—(C₁-C₁₂)-alkyl.

2. Compound according to claim 1,
wherein R¹ is selected from: —OCH₃, -^{tert}Bu.

3. Compound according to claim 1,
wherein R¹ is -^{tert}Bu.

4. Compound according to claim 1,
wherein R² is selected from: —OCH₃, -^{tert}Bu.

5. Compound according to claim 1,
wherein R² is -^{tert}Bu.

6. Compound according to claim 1,
wherein R² is —OCH₃.

7. Compound according to claim 1,
wherein R³ is selected from: —OCH₃, -^{tert}Bu.

8. Compound according to claim 1,
wherein R³ is -^{tert}Bu.

9. Compound according to claim 1,
wherein R⁴ is selected from: —OCH₃, -^{tert}Bu.

10. Compound according to claim 1,
wherein R⁴ is -^{tert}Bu.

11. Compound according to claim 1,
wherein R⁴ is —OCH₃.

12. Compound according to claim 1,
wherein the compound has the structure (1) or (2):

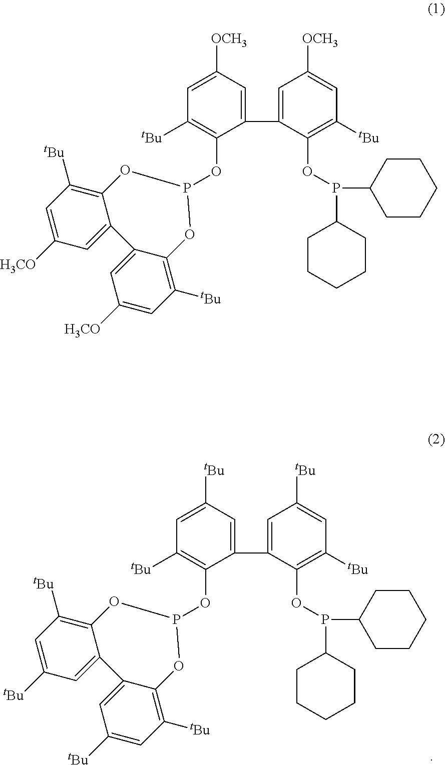 [SMILES: COc1cc(-c2cc(OC)cc(C(C)(C)C)c2Op2oc3c(C(C)(C)C)cc(OC)cc3c3cc(OC)cc(C(C)(C)C)c3o2)c(OP(C2CCCCC2)C2CCCCC2)c(C(C)(C)C)c1 ; CC(C)(C)c1cc(-c2cc(C(C)(C)C)cc(C(C)(C)C)c2Op2oc3c(C(C)(C)C)cc(C(C)(C)C)cc3c3cc(C(C)(C)C)cc(C(C)(C)C)c3o2)c(OP(C2CCCCC2)C2CCCCC2)c(C(C)(C)C)c1]

13. Process comprising the process steps of:
a) initially charging an olefin;
b) adding a compound according to claim 1;
c) adding a Rh compound;
d) feeding in H₂ and CO;
e) heating the reaction mixture from a) to d), to convert the olefin to an aldehyde.

14. Process according to claim 13,
wherein the Rh compound is selected from: Rh(acac)(CO)₂, [(acac)Rh(COD)] (Umicore, acac=acetylacetonate anion; COD=1,5-cyclooctadiene), Rh₄CO₁₂.

15. Process according to claim 13,
wherein the Rh compound is Rh(acac)(COD).
